(19) United States
(12) Patent Application Publication
(10) Pub. No.: US 2010/0204609 A1
(43) Pub. Date: Aug. 12, 2010

(54) MICROENDOSCOPE AND METHODS OF USE

(76) Inventors: Howard WORTH, Venice, CA (US); David Walker, Pasadena, CA (US)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 12/503,625

(22) Filed: Jul. 15, 2009

Related U.S. Application Data
(60) Provisional application No. 61/151,407, filed on Feb. 10, 2009.

(51) Int. Cl. A61B 10/04; A61B 1/00; A61B 1/07; A61B 1/018

(52) U.S. Cl. 600/562; 600/114; 600/182; 600/104

(57) ABSTRACT

The present invention generally relates to a microendoscope assembly capable of intra-ductal use. The microendoscope generally comprises: an outer cylindrical guide tube defining an internal passageway having an exterior diameter of no greater than about 1.0 mm; a first internal cylindrical tube defining a working channel having an interior diameter of no less than about 0.5 mm; a second internal cylindrical tube defining a image guide and housing a lens; and an illumination bundle comprising optic fibers.

### FIELD OF THE INVENTION

The present invention is generally directed toward a microendoscope and methods of use thereof.

### BACKGROUND OF THE INVENTION

In recent years there has been increasing use of endoscopes for minimally invasive treatment and diagnosis. For example, endoscopes have attempted to be used in diagnostic procedures, e.g., in sinus cavities, breast tissues, and other small diameter ducts. However, current endoscope designs are limited in the small spaces required.

Breast cancer is the second most common malignancy among women. In the United States alone: (a) one person dies from breast cancer every 14 minutes; (b) 250,000 cases of breast cancer are confirmed annually: (c) there is a lifetime risk that one in eight women will have cancer in their lifetime; (d) it is estimated that 2.6 million women have breast cancer; (e) 75% of breast cancers occur in women over 50 years of age; (f) breast cancer is the most common malignancy among women; (g) 40 million mammograms are performed annually; (h) 1.2 million biopsies are done annually with the National Cancer Institute predicting over 400,000 new cancer diagnoses annually by the year 2017; and (i) annually, worldwide, one million new cases of breast cancer are reported and over 500,000 women die as a result of the disease. In addition, there is a new diagnosis of breast cancer every 3 minutes with breast cancer being the most invasive cancer among women in United States. In 2008, it was estimated that over 250,000 new cases and over 40,000 died in the United States from breast cancer, with more than 1 in 3 cancers being breast cancer and 2.4 million women in having been diagnosed and treated for breast cancer. (Data obtained from NCI, Breast Cancer Network, Breast Cancer.Org, NIH, etc.)

Given the prevalence of breast cancer, the need for improved early detection of abnormal tissue, and the need for improved treatment options (e.g., biopsy, removal, brachytherapy, etc.)—improvements in microendoscopes for use in small tissues and ducts are desirable.

Thus, there is a need in the art for new and better microendoscopes and methods for using the same that can be used to directly visualize small tissue areas, e.g., mammary ducts of a breast, for diagnostic, e.g., detection of abnormal tissue, as well as therapeutic applications, e.g., brachytherapy. Additionally, there exists a need for microendoscopes that provide, simultaneously, both real-time visualization and a functional “working channel” for early diagnostics (e.g. biopsy) and that can additionally be used as a “delivery system” for localized and/or location-specific treatment and preventative methodologies.

All documents referred to herein are incorporated by reference into the present application as though fully set forth herein.

### SUMMARY OF THE INVENTION

In accordance with the present invention, a microendoscope assembly is provided. In general, the microendoscope comprises: an outer cylindrical guide tube defining an internal passageway having an exterior diameter of no greater than 1 mm; a first internal cylindrical tube defining a working channel having an interior diameter of no less than 0.5 mm; a second internal cylindrical tube defining a image guide and housing a lens; and an illumination bundle comprising optic fibers.

In certain aspects, because the cannula tube is of such a small outer diameter, the physician can manipulate the tube from the proximal end in order to guide the microendoscope to the desired location within a subject.

In certain embodiments, in operation of the microendoscope of the invention, the microendoscope may be placed, e.g., in a duct in a subject's breast with real-time visualization during placement. The physician can view the interior of the duct, as the endoscope passes on its way through the duct to the area of interest. Once at the duct area of interest, the physician can insert micro-instruments into working channel of the microendoscope, as desired while maintaining real-time imaging of the site of interest. When the manipulation of the micro-instrument is completed, the entire assembly may be removed from the duct and breast.

These and other aspects of the invention will be more clearly understood with reference to the following preferred embodiments and detailed description.

### BRIEF DESCRIPTION OF THE DRAWINGS

FIG. 1A illustrates a microendoscope assembly of the invention in side view.

FIG. 1B illustrates a microendoscope of the invention in cross-section.

### DETAILED DESCRIPTION OF THE INVENTION

The present invention generally relates to a microendoscope assembly capable of intra-ductal use. The microendoscope generally comprises: an outer cylindrical guide tube defining an internal passageway having an exterior diameter of no greater than about 1.0 mm; a first internal cylindrical tube defining a working channel having an interior diameter of no less than about 0.5 mm; a second internal cylindrical tube defining a image guide and housing a lens; and an illumination bundle comprising optic fibers.

The microendoscope of the invention allows for minimally invasive procedures, e.g., in the physician's office or outpatient setting, without the trauma typically associated with surgery. In additional, the microendoscope provides for real time intra-ductal diagnostic imaging, e.g., within the breast duct and provides a visually assisted “working channel” within the scope. In certain embodiments, the working channel provides a “delivery system” for using micro-tools for both treatment and prevention. For example, the working channel provides the opportunity to make precision-targeted tissue removal for biopsy and analysis.

In accordance with certain aspects of the invention, it has been surprisingly found that the microendoscope of the invention provides a minimally-invasive method for detecting early-stage breast cancer in advance of conventional methods, e.g., five (5) to seven (7) years before conventional mammography is capable of identifying such small growths. (Conventional mammography references thanks to Dr. William Dooley, surgical oncologist, breast cancer specialist). The microendoscope of the invention provides intra-ductal real-time imaging, e.g., within the breast ducts, thereby illuminating and magnifying sub-millimeter growths that are identifiable as early-stage cancer or pre-cancerous.

In addition to the ability of the microendoscope to visually identify papillomas, lesions, and other anomalies within the breast ducts, the microendoscope of the present invention comprises a working channel of no less than about 0.5 millimeter (wherein the outer diameter of the microendoscope is not greater than about one millimeter). This combination allows a practitioner to maintain constant visualization within the duct so as to locate, identify, and target very specific tissue, growths or lesions, etc., and then precisely excise, manipulate, or otherwise treat said tissue, growths or lesions with micro-instruments from within the working channel.

Prior technology allowed visualization within, e.g., a breast endoscope. However, at the point in time a sample was desired, two major inadequacies occurred: (a) the optical elements had to be removed and thus video imaging was lost entirely; and (b) the capability for maneuvering and operating micro-instruments to precise destinations in order to excise the targeted tissue was not possible. This meant that any tissue sampling was mainly retrieved randomly from already dislodged material that arbitrarily happened to being “floating” in the duct at that given moment, or at best, sampling was obtained “in the dark.” Because biopsy results are limited by the quality of material which is presented for analysis due to the inability to differentiate, target and acquire optimized biological material, the likelihood of accurate biopsy results is substantially reduced or negated entirely.

A. Microendoscope and Exemplary Use

As mentioned above, in a first aspect the present invention is directed towards a microendoscope assembly 10 capable of intra-ductal use. In certain embodiments, the microendoscope assembly 10 is configured for use in breast ducts.

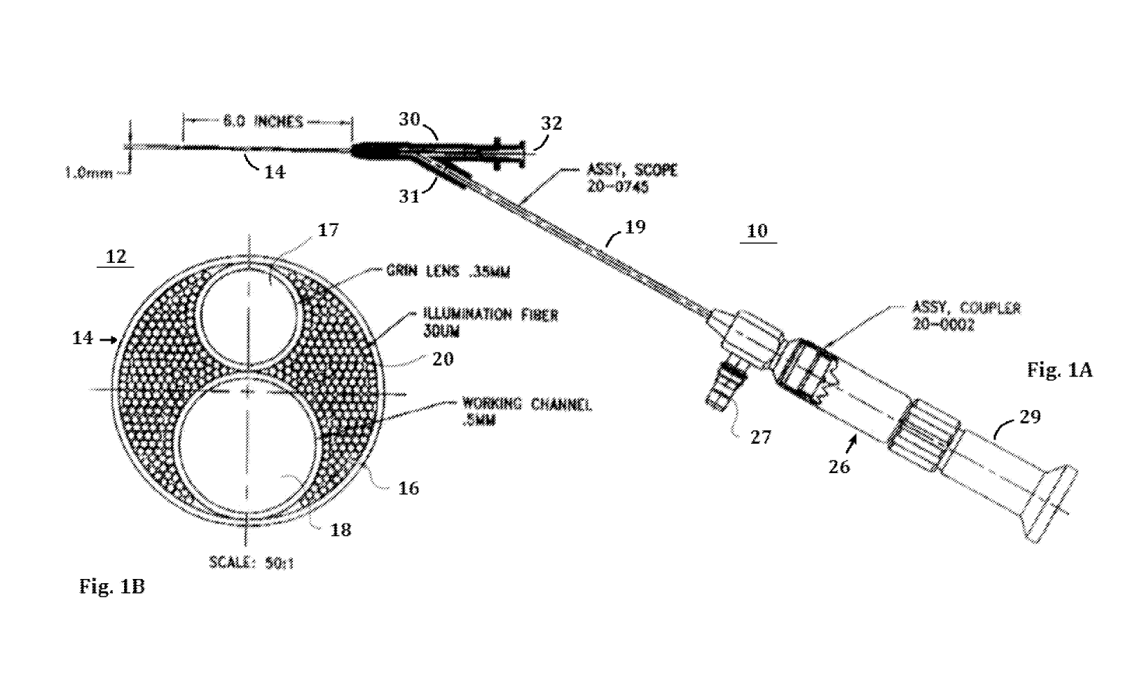

With reference to FIGS. 1A and 1B, the microendoscope assembly 10 includes an outer tube or guide cannula 14 which seats and guides the microendoscope 12. The cannula 14 has an outer cylindrical wall 16 which defines an internal passageway which runs along its length to seat and guide the microendoscope 12. Cannula 14 may be formed from any suitable material and may be any suitable length for its desired purpose, as recognized by those skilled in the art. For instance, it may range from about 5-20 cm long, preferably about 15.2 cm.

Further, in certain embodiments, cannula 14 is preferably formed from a flexible or semi-rigid material to aid in manipulation during use, e.g., a suitable polymer material may be used such as a polyimide. If additional rigidity is desired, wires may be added to the working channel by a practitioner. Changing the gauge or number of wires may alter the rigidity of the endoscope. If desired, the wires may be added after the microendoscope assembly is placed in the desired location within a subject so as to maximize flexibility. As mentioned above, the outer cylindrical wall 16 of cannula 14 has an outer diameter of not greater than about 1.0 mm. In certain embodiments, the outer diameter may range from 0.9 mm to 1.0 mm, 0.95 mm to 1.0 mm, or 0.98 mm to 1.0 mm.

In certain embodiments, the exterior of the cannula may be marked with marking indicia (not shown) so that the depth of penetration of the microendoscope assembly into a duct can be noted. The marking indicia can be in the form of rings of opaque, translucent or light reacting material or any other suitable geometry which is easily visible to the surgeon's eye, as generally recognized by those skilled in the art. The marking indicia can be printed onto the outer surface of the cannula or imbedded in the cannula structure material. Various cannulae are envisioned to be interchangeable with the endoscope 12 by unscrewing one guide cannula from the endoscope front hub 30 and its associated connector member and screwing one another on to the connector member.

The microendoscope 12 is provided with a first internal cylindrical tube defining a working channel 18, and a second internal cylindrical tube body defining an image guide 17 formed with a lens at its distal end. As mentioned above, the working channel 18 has an interior diameter of no less than about 0.5 mm. In certain embodiments, the interior diameter ranges from 0.5 mm to 0.6 mm, 0.5 mm to 0.55 mm, or 0.5 mm to 0.52 mm. Any suitable image guide and lens may be used to provide the desired image quality, as recognized by those skilled in the art. By way of example, a 6000 pixel image guide and a grin lens (e.g., a 0.35 grin lens) may be used. Again, the interior cylindrical tubes may be formed from any suitable material known in the art, and are preferable flexible or semi-rigid materials such as polymers, e.g., polyimides.

The microendoscope assembly 10 has a proximal end in the form of a back member 26 having a light post 27 and a video port/eye piece 29. Microendoscope assembly 10 also includes hub 30 at a proximal end to couple back member 26 to guide cannula 14. Hub 30 includes image coupler 31 and working channel coupler 32. In use, a practitioner may insert micro-instruments into the working channel 18 via working channel coupler 32.

Microendoscope 12 also includes space in its interior capable of holding fiber optic strands and/or illumination strands 20. Such strands run from light post 27, through tube portion 19 into hub 30. The strands 20 run through hub 30 into the inner passageway of the interior of microendoscope 12. These strands provide a light source to the area of interest, allowing the physician to see an image of the area of the interest to add to the uniqueness to our scope's placement of the fibers within the scope, (different than other scopes) based on the drawings. Excellent results were obtained with the optic fiber strands occupying the space between the internal cylindrical tubes and the inner wall of the outer guide tube, however the optic fibers can suitably arranged in various fashions. Any suitable optic or illumination fibers may be used, e.g., an illumination bundle 0.012 in diameter including approximately 103/30 micron fibers with a 0.66 na.

Because the cannula tube is of such a small outer diameter, the physician can manipulate the tube from the proximal end in order to guide the microendoscope to the desired location within a subject. In certain embodiments, in operation of the microendoscope assembly 10, the guide cannula 14 is placed in a duct in a subject's breast. The physician can view the interior of the duct via video port/eye piece 29, as the endoscope passes on its way through the duct to the area of interest. Once the duct area of interest, the physician can insert micro-instruments into working channel 18 as desired while maintaining real-time imaging of the site of interest. When the manipulation of the micro instrument is completed, the entire assembly is removed from the duct and breast.

B. Additional Uses

In a first aspect, the microendoscope assembly of the present invention may be used in breast cancer diagnostics using the working channel of the microendoscope to identify and retrieve targeted tissue samples for biopsy. The present invention allows for contemporaneous, real-time imaging during sampling through the working channel.

In another aspect, the microendoscope assembly of the present invention may be used such that the internal working channel can provide access to practice a variety of surgical procedures on cells, tissue and organs at specially located particular sites. As described above, the microendoscope assembly of the invention allows for concurrent real-time visualization of the surgical site via the image guide during use of the working channel.

In another aspect, the microendoscope assembly of the present invention may be used in breast cancer treatment using the scope/working channel delivery system for targeted bracbo brachytherapy with contemporaneous, real-time imaging during placement.

In another aspect, the microendoscope assembly of the invention may be used in breast cancer prevention using the scope/working channel as a delivery system to deliver a marker or “tag” to an area of significance, e.g., locations where growths are observed, biopsied, excised, etc. Once the marker system is in use, the breast ducts can be “mapped” for treatment/prevention and tracked for success or otherwise. In this way, a 3D “database” for a patient can be developed for optimal treatment over time.

In other aspects of the invention, the microendoscope assembly may be configured for use in any intra-duct application or other use where access to small areas (less than about 1 mm) is desirable. Such areas include sinus, blood vessels, the gastrointestinal tract, optical applications, cerebral applications, etc. Specifically, methods and assemblies as described above may be used in applications including cerebral application including the globe, ventricles of the brain, subarachnoid space around the brain; sinus-related applications including maxillary, sphenoid ethmoid sinuses and frontal sinus duct, pathologic fistula or sinus tracts i.e. enterocutaneous fistula, bronchial cleft remnants, endoscopic ethmoidectomy, maxillectomy and frontal sinus cannulation; digestive excretory ducts; middle ear cavity for ossicular visualization. In addition, the microendoscope may be utilized in optical applications, e.g., iatrogenic potential optical cavities, and for diagnosing and treating various blood vessel disorders.

Additional uses include endoscopy in small joint spaces (i.e. TMJ joint, interphalangeal joints, wrist joints, ankle joints, etc.); endoscopy in minimally invasive plastic surgery—brow, facelift, breast augmentation etc., nasopharyngoscopy and microlaryngoscopy, adjunct microendoscopy for TMJ surgery, reduction and fixation of maxillifacial and mandibular fractures (especially zygomatic arch, orbital floor and subcondylar fractures). The microendoscope assembly of the invention may also be used in trans g-tube and j-tube inspections; in adjunct visualization of cavity with drain, shunt, etc.; in adjunct with microlaminectomy for intervertebral space; and as adjunct with surgical navigation, neurosurgery, ENT, spine, etc. The microendoscope assembly can also be used for particular delivery of chemotherapeutic agents or electromagnetic radiation, such as laser, microwave and high-energy waves, for treatment to specifically located sites, utilizing the internal working channel for such delivery.

### EXAMPLES

The present invention is described in more detail with reference to the following non-limiting examples, which are offered to more fully illustrate the invention, but are not to be construed as limiting the scope thereof. The principles, embodiments and modes of operation of the present invention have been described in the foregoing specification. For example, the present invention is not limited to the particular dimensions, materials, or uses described, except as explicitly defined in the claims. Instead, the embodiments described here should be regarded as illustrative rather than restrictive. Variations and changes may be made by others without departing from the scope of the present invention as defined by the following claims.

### Example

A microendoscope assembly as outlined in FIGS. 1A-1B was developed and tested. Results of the testing are summarized below.

I) IMAGE SIZE: Increased significantly over earlier Generations. (68% screen coverage of a 19″ monitor was routinely produced.)

II) IMAGE QUALITY: An 8″ screen image consistently yielded great quality clarity, color and depth of field.

III) CONTINUOUS IMAGING: Constant real-time video imaging was maintained continuously without distortion.

IV) MANEUVERABILITY FACTOR: Highly flexible shaft allowed significantly increased “corner turning” ability and mobility around obstacles.

V) IMMERSION FUNCTIONALITY: Scope performed under liquids of varying density, maintaining benchmark image quality through the water whether standing, swirling or other turbulence.

All publications and patent applications cited herein are incorporated by reference to the same extent as if each individual publication or patent application was specifically and individually indicated to be incorporated by reference.

Although certain embodiments have been described in detail above, those having ordinary skill in the art will clearly understand that many modifications are possible in the embodiments without departing from the teachings thereof. All such modifications are intended to be encompassed within the claims of the invention.

What is claimed:

1. A microendoscope assembly comprising:
an outer cylindrical guide tube defining an internal passageway having an exterior diameter of no greater than 1 mm;
a first internal cylindrical tube defining a working channel having an interior diameter of no less than 0.5 mm;
a second internal cylindrical tube defining a image guide and housing a lens; and
an illumination bundle comprising optic fibers.

2. A microendoscope assembly of claim 1, wherein the image guide is a 6000 pixel image guide.

3. A microendoscope assembly of claim 1, wherein the lens is a grin lens.

4. A microendoscope assembly of claim 1, wherein the outer cylindrical tube, the first internal cylindrical tube and the second internal cylindrical tube are each formed from semi-rigid or flexible polymer materials.

5. A microendoscope assembly of claim 4, wherein the polymer material is a polyimide.

6. A microendoscope assembly of claim 1, further comprising:
a hub at a proximal end of the outer cylindrical guide tube, the hub having an image coupler and a working channel coupler; and
a back member proximal to and interfaced with image coupler of the hub, the back member having a light post and a video port/eye piece;
wherein the optic fibers of the illumination bundle pass through the hub, the image coupler and to the light post of the back member.

7. A method of using a microendoscope assembly of claim 1 in breast cancer diagnostics in a subject in need thereof using the working channel of the microendoscope to identify and retrieve targeted tissue samples for biopsy, the method comprising:
guiding the microendoscope to a desired location within a duct of a subject's breast while viewing the interior of the duct via image guide and lens as the endoscope passes on its way through the duct to the desired location;
inserting micro-instruments into the working channel of the microendoscope while maintaining real-time imaging of the desired location via the image guide and lens;
manipulating the micro instrument to retrieve a targeted tissue sample for biopsy; and
removing the entire microendoscope assembly from the duct and breast with the targeted tissue sample.

8. A method of using a microendoscope assembly of claim 1 in intra-ductal endoscopy in a subject in need thereof, the method comprising:
guiding the microendoscope to a desired location within a duct having a diameter less than about 1 mm of a subject while viewing the interior of the duct via image guide and lens as the endoscope passes on its way through the duct to the desired location;
inserting micro-instruments into the working channel of the microendoscope while maintaining real-time imaging of the desired location via the image guide and lens;
manipulating the micro-instrument in a desired manner to achieve a desired result within the duct; and
removing the entire microendoscope assembly from the duct.

9. The method of claim 8, wherein the endoscopy procedure is a biopsy, and the micro-instrument is manipulated to as to retrieve a targeted tissue sample for biopsy.

10. The method of claim 8, wherein the endoscopy procedure is a microsurgery, and the micro-instrument is manipulated to as repair a targeted tissue.

11. The method of claim 8, wherein the duct location is selected from the group consisting of: breast ducts, sinus cavities, blood vessels, the gastrointestinal tract, optical space, and cerebral spaces.
